(19) United States
(12) Patent Application Publication
(10) Pub. No.: US 2026/0056125 A1
(43) Pub. Date: Feb. 26, 2026

(54) BIOFILM DETECTION AND SANITIZATION VIA FLUORESCENCE USING A FLEXIBLE SERPENTINE DEVICE

(71) Applicant: INTERNATIONAL BUSINESS MACHINES CORPORATION, Armonk, NY (US)

(72) Inventors: Heather Nicole Polgrean, Lagrangeville, NY (US); John S. Werner, Fishkill, NY (US); Jeremy R. Fox, Georgetown, TX (US); Jessica Nahulan, Markham, CA (US); Tyler Hansen, Tucson, AZ (US)

(21) Appl. No.: 18/809,834

(22) Filed: Aug. 20, 2024

(51) Int. Cl. G01N 21/64; A61L 2/08

(52) U.S. Cl. CPC G01N 21/645; A61L 2/084; G01N 2021/6417; G01N 2021/6484; G01N 2201/062

(57) ABSTRACT

Systems and methods for detecting and sanitizing biofilms are disclosed. A device for biofilm detection comprises a plurality of sensing nodes, each of the plurality of sensing nodes comprising: one or more probes spaced around and defining a center lumen of the sensing node and having a surface directed outwardly from the center lumen, each of the one or more probes comprising: a quartz window disposed in the surface; and a fiber optic element disposed in optical communication with the quartz window; one or more guide wire ports running parallel to the center lumen; a plurality of swivel joints, each of the plurality of swivel joints interconnecting adjacent pairs of the plurality of sensing nodes; and one or more guide wires extending through the guide wire ports.

### TECHNICAL FIELD

Embodiments of the present disclosure are related to an apparatus and method of use for a biofilm detection and sanitization device.

### BACKGROUND OF THE DISCLOSURE

A biofilm is a structured community of microorganisms, including bacteria and fungi, encased in a self-produced extracellular matrix. Biofilms form on surfaces in aqueous or humid environments, such as pipes, tanks, ventilation, and industrial equipment.

Biofilms pose a significant risk to a number of industries, including the semiconductor, healthcare, and water sanitation industries. As a contamination risk, biofilm can harbor and release contaminants, including particles, chemicals, and organic matter, which can negatively impact manufacturing processes, water purification, and other sterile environments. Biofilm matrices can cause physical damage to manufactured surfaces, potentially leading to defects and reduced product yield. Further, clogged pipes and filters due to biofilm growth can reduce the efficiency of cooling systems and water purification processes crucial to fabrication and manufacturing processes. Biofilms can promote chemical corrosion in equipment, leading to maintenance issues and costly repairs. Additionally, microbes in biofilms can produce byproducts that contribute to defects in manufactured devices, such as semiconductors. Biofilms on medical equipment, such as catheters and implants, can lead to infections and patient complications. Contamination due to biofilms can potentially lead to foodborne illnesses and product recalls in the food processing setting, as well as leaks and environmental damage due to corroded pipelines, tanks, and other equipment. Biofilms can interfere with water treatment processes, reducing water quality and increasing costs.

An effective biofilm detection and sanitization device, able to flexibly move throughout a variety of environments, is needed.

### SUMMARY

According to aspects of the present disclosure, an apparatus and methods of using are disclosed for a biofilm detection and sanitization via fluorescence using a flexible serpentine device.

In one embodiment, a biofilm detection device comprises: a plurality of sensing nodes, each of the plurality of sensing nodes comprising: one or more probes spaced around and defining a center lumen of the sensing node and having a surface directed outwardly from the center lumen, each of the one or more probes comprising: a quartz window disposed in the surface; and a fiber optic element disposed in optical communication with the quartz window; one or more guide wire ports running parallel to the center lumen; a plurality of swivel joints, each of the plurality of swivel joints interconnecting adjacent pairs of the plurality of sensing nodes; and one or more guide wires extending through the guide wire ports.

In some embodiments, the biofilm detection device further comprises a hollow backbone carrying fiber optic cabling from the sensing nodes in communication with a control unit.

In some embodiments, the probes are air- or water-tight.

In some embodiments, the quartz window comprises a UV grade quartz, and transmits UV light ranging from 180 nm to 400 nm.

In some embodiments, the one or more probes further comprises one or more light emitting diodes (LED).

In some embodiments, each sensing node of the plurality of sensing nodes comprises a plurality of optically transparent windows configured to provide 360-degree light emission and sensing.

In some embodiments, each sensing node of the plurality of sensing nodes is independently operative.

In some embodiments, each sensing node of the plurality of sensing nodes further comprises a series of open ports positioned symmetrically about the center lumen.

In some embodiments, each quartz window is detachably attached to one or more of the open ports by a latch.

In some embodiments, the fiber optic element is configured to receive emitted fluorescence from biofilm.

In some embodiments, the plurality of sensing nodes is configured to emit fluorescence to thereby sanitize biofilm.

In some embodiments, the plurality of sensing nodes is operatively connected to a control unit via one or more wires extending through the center lumen.

In some embodiments, the swivel joints comprise hollow ball joints.

In some embodiments, the plurality of sensing nodes is operatively connected to a control unit via a wireless network.

In an alternative embodiment, a method of detecting biofilm comprises activating one or more sensing nodes on a biofilm detection device, the biofilm detection device comprising at least one sensing node with an illumination source; scanning an entity using the biofilm detection device; detecting a threshold level of a biofilm within the entity; determining a wavelength and an illumination duration for the threshold level of the biofilm; illuminating, by the illumination source, a location of biofilm within the entity for a determined illumination duration; determining the biofilm is removed from the location; and outputting a report of an end level of biofilm within the entity.

In some embodiments, wherein the flexible UV fluorescence biofilm detection device comprises: a plurality of sensing nodes, each of the plurality of sensing nodes comprising: one or more probes spaced around and defining a center lumen of the sensing node and having a surface directed outwardly from the center lumen, each of the one or more probes comprising: a quartz window disposed in the surface; and a fiber optic element disposed in optical communication with the quartz window; one or more guide wire ports running parallel to the center lumen; a plurality of swivel joints, each of the plurality of swivel joints interconnecting adjacent pairs of the plurality of sensing nodes; and one or more guide wires extending through the guide wire ports.

In some embodiments, the method further comprises determining a biofilm remains in the location; illuminating, by the illumination source, a location of biofilm for an additional illumination duration; determining the biofilm is removed from the location; and updating the report.

In some embodiments, the method further comprises updating a database associated with the entity, wherein the database includes changes made to eliminate biofilm within the entity.

In some embodiments, the method further comprises communicating the report to a display.

In some embodiments, scanning the entity comprises a lateral, 360-degree biofilm sensing and sanitizing by the one or more nodes.

In some embodiments, the method further comprises inducing a fluorescent emission from the biofilm by the one or more nodes.

In some embodiments, the method further comprises storing and recording the fluorescent emission as the entire entity is scanned.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a schematic overview of an embodiment of a flexible serpentine device, in accordance with one or more embodiments of this disclosure.

FIG. 2A is a schematic view of a node of the flexible serpentine device, in accordance with one or more embodiments of this disclosure.

FIG. 2B is a schematic view of a node of the flexible serpentine device, in accordance with one or more embodiments of this disclosure.

FIG. 3 is a schematic view of an exemplary probe of the node, in accordance with one or more embodiments of this disclosure.

FIG. 4 is a schematic view of an exemplary backbone of the node, in accordance with one or more embodiments of this disclosure.

FIG. 5 is a top view of the exemplary backbone of the node, in accordance with one or more embodiments of this disclosure.

FIG. 6 is an illustration of an exemplary guide wire, in accordance with one or more embodiments of this disclosure.

FIG. 7 is is a schematic view of an exemplary power and control unit, in accordance with one or more embodiments of this disclosure.

FIG. 8 is flow diagram illustrating a method of use of the flexible serpentine device, in accordance with one or more embodiments of this disclosure.

FIG. 9 is an illustration of an exemplary device used in combination with a holding tank, in accordance with one or more embodiments of this disclosure.

FIG. 10 is an illustration of an exemplary device used in a medical procedure, in accordance with one or more embodiments of this disclosure.

FIG. 11 is a schematic view of an exemplary computing node.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

A biofilm may comprise any syntrophic consortium of microorganisms in which cells stick to each other and often also to a surface. These adherent cells become embedded within a slimy extracellular matrix that is composed of extracellular polymeric substances (ESPs). The cells within the biofilm produce the EPS components, which are typically a polymeric combination of extracellular polysaccharides, proteins, lipids and DNA. Because they have a three-dimensional structure and represent a community lifestyle for microorganisms, they have been metaphorically described as “cities for microbes.”

Biofilms may form on living (biotic) or non-living (abiotic) surfaces and can be common in natural, industrial, and hospital settings. They may constitute a microbiome or be a portion of it. The microbial cells growing in a biofilm are physiologically distinct from planktonic cells of the same organism, which, by contrast, are single cells that may float or swim in a liquid medium. Biofilms can form on the teeth as dental plaque, where they may cause tooth decay and gum disease.

Microbes form a biofilm in response to a number of different factors, which may include cellular recognition of specific or non-specific attachment sites on a surface, nutritional cues, or in some cases, by exposure of planktonic cells to sub-inhibitory concentrations of antibiotics. A cell that switches to the biofilm mode of growth undergoes a phenotypic shift in behavior in which large suites of genes are differentially regulated.

A biofilm may also be considered a hydrogel, which is a complex polymer that contains many times its dry weight in water. Biofilms are not just bacterial slime layers but biological systems; the bacteria organize themselves into a coordinated functional community. Biofilms can attach to a surface such as a tooth or rock, and may include a single species or a diverse group of microorganisms. Subpopulations of cells within the biofilm differentiate to perform various activities for motility, matrix production, and sporulation, supporting the overall success of the biofilm. The biofilm bacteria can share nutrients and are sheltered from harmful factors in the environment, such as desiccation, antibiotics, and a host body's immune system. A biofilm usually begins to form when a free-swimming, planktonic bacterium attaches to a surface.

To effectively detect and remove biofilm, embodiments of a flexible UV fluorescence biofilm detection system is disclosed herein. Embodiments of the present disclosure include multiple individual sensing elements or nodes, wherein each node is capable of providing a lateral, 360-degree, biofilm sensing, and sanitization ability. Multiple nodes may be grouped together to provide a three-dimensional sensing ability, enabling quick inspection of a larger area. Alternatively, an individual node or nodes can be selected and activated as desired within the system.

Each node may be associate with one or more open ports, where the open ports are positioned symmetrically along the diameter of a single node. The nodes may be housed within individual air- and/or water-tight probes having UV grade quartz windows (meaning UV transparent quartz), or windows of a similar UV transparent material, attached to the open ports using a docking base and a latching mechanism. The probes consist of at least one or more fiber optic elements that help to achieve several objectives: fluoroscopic sensing of biofilm; detection of emitted fluorescence from biofilm; and sanitization of biofilm. In another embodiment, the individual probes may consist of single or multiple light emitting diodes capable of achieving the aforementioned objectives.

In some embodiments, induced fluorescence captured from a biofilm, by individual nodes, is stored and recorded as the entire system is swept or scanned across the surface of interest. The fluorescent light may be output from an light emitting diode (LED) source operating in a specific wavelength, and may shine through one or more ports within the node.

In some embodiments, individual nodes are connected to each other using a swivel hollow ball joint.

The flexible serpentine device may include one or more guide wires, a flexible hollow backbone, and a power and control unit. The guide wires may be utilized for steering individual elements in the vertical and horizontal directions. To accommodate guide wires, each individual node may include a through hole for each of the guide wires. Each guide wire may be a metal wire surrounded by a spring with a certain degree of flexibility and stiffness. This helps to ensure that the device returns to its home position following a direction change. The guide wires may be connected to a industry standard mechanical steering mechanism utilizing a joystick. In some embodiments, an automated steering mechanism is envisioned as well.

The flexible hollow backbone may act as a central conduit for various fiber optic probe cabling, related wiring, etc., and may be connected to individual nodes using dual tresses. It is contemplated that the backbone contains the necessary cut outs that allows the individual fiber optic probe cabling and related wiring to be connected to various ports located on the outer surface of the nodes.

The power and control unit may be equipped with light emitting diodes sources and narrow band pass filters for producing and capturing UV light with a desired wavelength or wavelengths. In some embodiments, the power and control unit may include photomultiplier tubes for enhancing the emitted or captured signal, etc. It is contemplated that the power and control unit is capable of changing the wavelength of the UV light produced for killing biofilm and sanitizing surfaces, as well as being equipped with controls for manipulating the guide wires. In one embodiment, the guide wire control may be performed using a joystick positioned on the control unit, and in some embodiments, a camera may be positioned at the end of the device for visual inspection purposes.

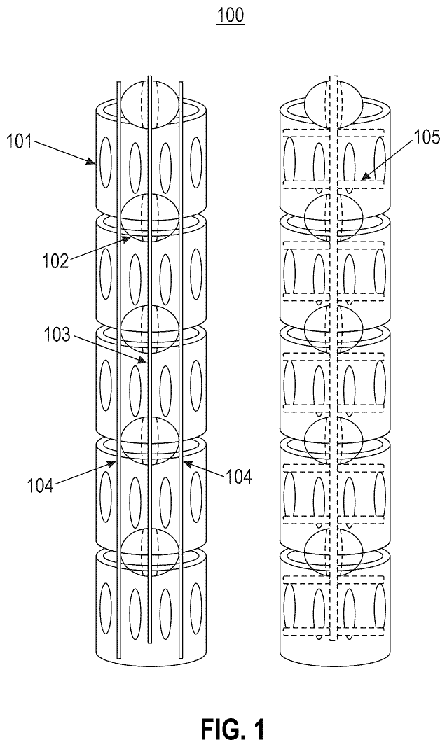

FIG. 1 is an overview of an embodiment of the flexible serpentine device 100. Device 100 comprises a number of nodes 101, a swivel hollow ball joint 102, a hollow backbone 103, at least one guide wire 104, and a backbone truss 105. Individual nodes 101 contain air-tight probes enabling a lateral 360 degree sensing and sanitization capability. Each individual node is physically connected to each other using swivel hollow ball joints 102 and can enable three dimensional biofilm sensing and sanitizing capacities.

Guide wires 104 and swivel hollow ball joints 102 provide the control element necessary for guiding the entire device through structures that possess bends and turns. The guide wires 104 and swivel hollow ball joints 102 allow for the serpentine motion of the device. The backbone truss 105 prevents deformation of the device.

The device 100 may be customized for a desired length, including as many nodes 101 as needed. In some embodiments, the nodes 101 may be interchangeable.

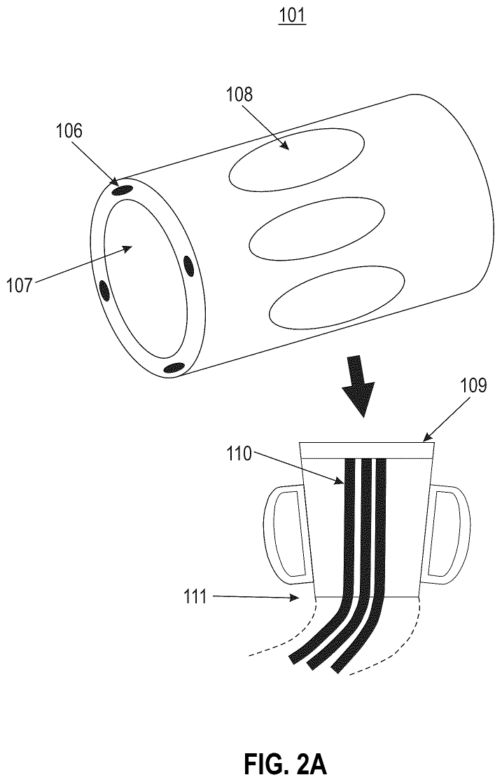

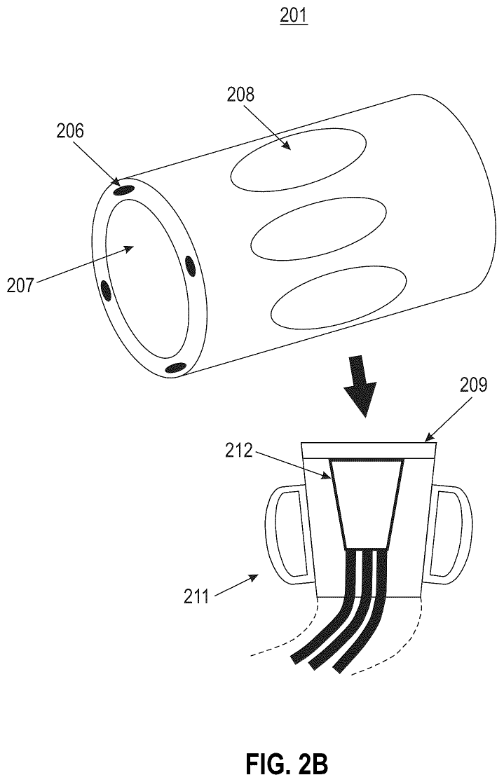

FIG. 2A illustrates an embodiment of an individual node 101. The node 101 is a hollow element which houses multiple probe elements, including guide wire ports 106, the defined hollow 107 of the node 101, as well as guide wires 104 and open ports 108 for attaching probes and additional components. In some embodiments, the node 101 is air- or water-tight. This may be particularly useful for deployment within industrial or manufacturing processes. Each node is utilized to sense the biofilm using UV fluorescence and to sanitize the biofilm. In some embodiments, the individual probes possess a quartz window comprised of UV grade quartz that enables transmission of UV light ranging from 180 nm to 400 nm. Other embodiments may contain other suitable UV grade materials that enable transmission. Bandpass filters may also be utilized for producing and sensing the UV light with the desired wavelengths.

Fiber optic probe 111 includes a UV quartz window 109 with one or more fiber optic elements 110. In some embodiments, the individual fiber optic probes 111 are removeable, and are secured to open ports located on the nodes when attached to the device. In the embodiment illustrated in FIG. 2A, only three fiber optic elements 110 of the fiber optic probe are shown. In another embodiment, multiple (i.e., greater than three) fiber optic elements may be utilized. It is contemplated that fewer than three elements may be used as well. In alternative embodiments, the individual probes may consist of single or multiple LEDs.

FIG. 2B illustrates an alternative embodiment of an individual node 201. The node 201 is still a hollow element which houses multiple probe elements, including guide wire ports 206, the defined hollow 207 of the node 201, as well as guide wires 204 and open ports 208 for attaching probes and additional components. In some embodiments, the node 201 is air- or water-tight. Notably, the embodiment illustrated in FIG. 2B includes a probe 211 having an LED encapsulated in the port 206. The fiber optic elements 110 as illustrated by FIG. 2A are replaced by a single LED 210 in the individual node 201 as shown in FIG. 2B. LED 210 may include multiple LEDs encapsulated in the port 206.

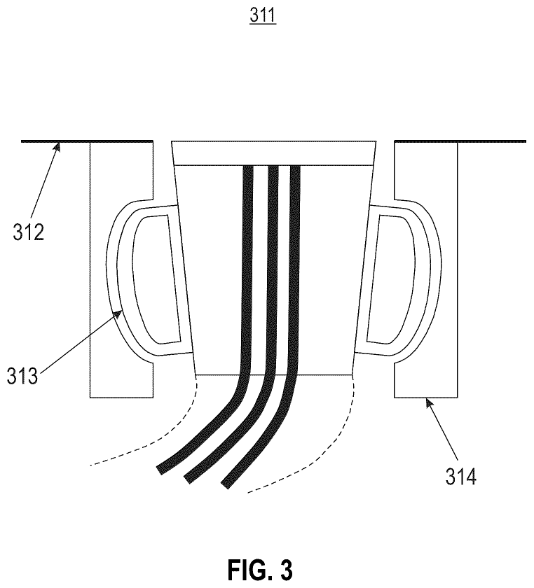

FIG. 3 illustrates an embodiment of an exemplary probe 311. The probe 311 is designed to be interchangeable within the node 101. Probe 311 may be docked such that the surface of the probe is aligned with the surface 312 of the associated node. Multiple open ports 108 may be located around a center hollow 107 of each node 101, allowing for a corresponding number of probes 311 to be deployed within the node. Each open port 108 may include a docking base 314 to which the probe 311 attaches. In one embodiment, a compressible latch mechanism 313 may be utilized to secure the probe 311 to the open port 108.

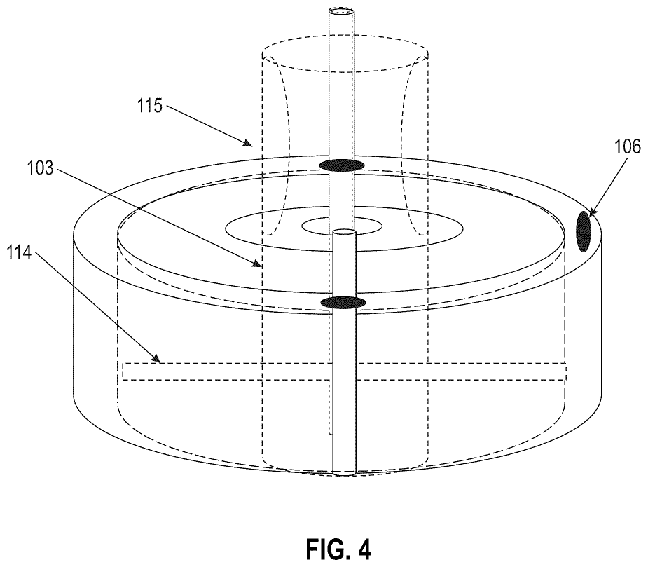

FIG. 4 illustrates an exemplary backbone 103 of the node 101. The hollow backbone 103 extends through the various nodes 101, allowing for the device 100 to have a great degree of flexibility while maintaining its rigid structure. The hollow backbone 103 allows each individual node 101 to pass through a narrow space while lending structural support to maintain a desired shape for navigation. A flexible rod or other suitable material may be connected to the interior of the hollow backbone 103 via one or more trusses 114. The trusses 114 may be arranged in any suitable arrangement within each node 101. The backbone truss 114 may comprise any suitable material, including a non-conductive alloy that maintains the device's structural integrity. The hollow backbone 103 may include one or more open ports 115, wherein each open port serves to allow access to the hollow backbone 103. This may be particularly useful where the nodes 103 are interchangeable, and may allow for the hollow backbone 103 to connect and/or disconnect from additional nodes 101. Additional support for the node 101 may be found in the guide wires running through guide wire ports 106 located circumferentially around the hollow backbone 103.

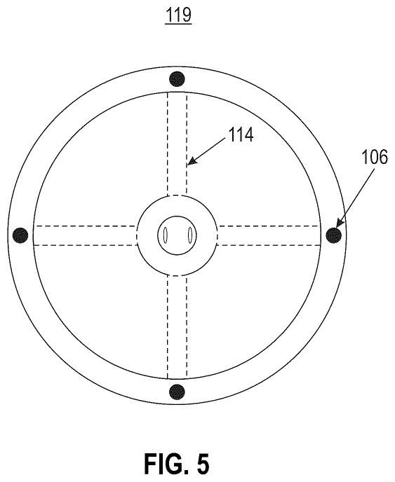

FIG. 5 is a cross section of the swivel hollow ball joint 119 The swivel hollow ball joint 119 connects the various nodes, enabling a three-rotational degree freedom of motion between each individual node. Each swivel hollow ball joint 119 may include one or more guide wire ports 106, as well as a backbone truss 114. The swivel hollow ball joint 119 may provide a connection to interchangeable nodes 101 situated above or below the ball joint, in some embodiments.

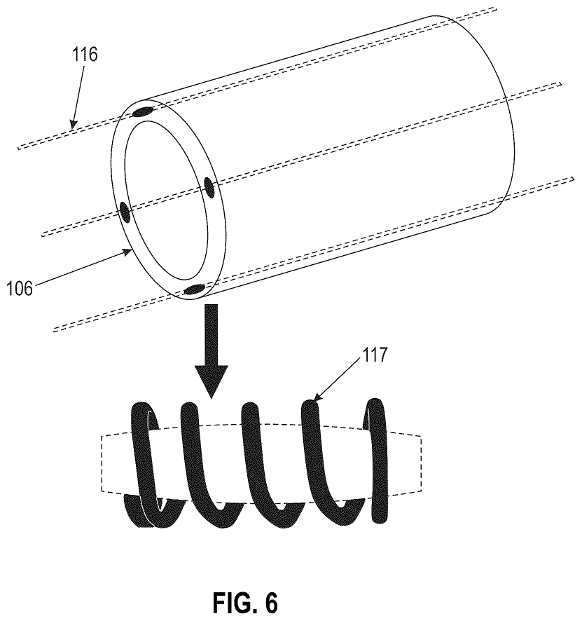

FIG. 6 illustrates the guide wires 116 running through the guide wire ports 106 in each node. Each guide wire 116 is utilized for steering individual node elements in the vertical and horizontal direction. Individual nodes 101 include guide wire ports 106, situated around the defined hollow of the node 101, which allow the guide wire 116 to run through and support the node 101. Each individual guide wire 116 comprises a metal wire surrounded by a spring 117 with a certain degree of flexibility and stiffness, as shown in the inset image of FIG. 6. The spring 117 helps to ensure the nodes 101 of the device 100 will return to their home positions following a directional change in the motion of the device 100. It is contemplated that the guide wires 116 are connected to an industry standard mechanical steering mechanism utilizing a joystick. In some embodiments, an automated steering mechanism is envisioned. The steering mechanism may be connected to a control unit, which may control the motion of the guide wires 116 when each guide wire is associated with a motor or other means of locomotion.

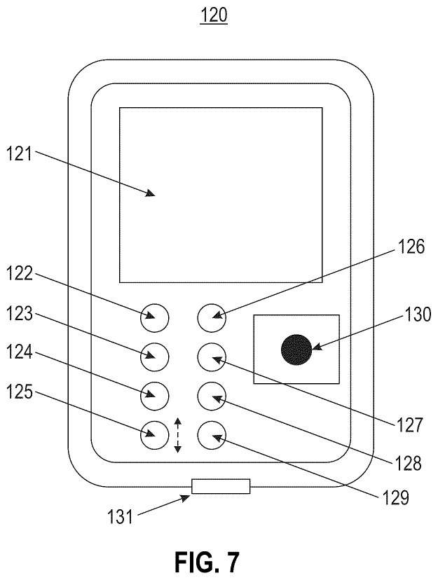

FIG. 7 illustrates a power and control unit 120. The power and control unit 120 may communicate with the device 101, thereby controlling the device 101 movement and biofilm sensing capacity as the device 101 is deployed. The power and control unit 120 may be wireless or wired, wherein HDMI port 131 can provide an input for digital communication if desired. The readout display screen 121 may have touch screen capability. The readout display screen 121 may display a single value from a node reading, or a graphic illustrating a biofilm location, thickness, consistency, or turbidity over time. The user may select any one of buttons 122-129, including detection 122, individual node selection 124, all nodes selection 123, UV LED illumination control 125, data save 126, data transfer 127, sanitization 128, and/or an image capture selection 129. Image capture selection 129 may be used in embodiments of device 101 including a camera or other image sensing apparatus on node 101. The power and control unit 120 may further comprise a joystick 130 for controlling the device 100 movement, wherein joystick motion may control the motion of the guide wires 116.

In some embodiments, the display screen 121 may display a map or a single quantity associated with a detected biofilm. The display may include large readings covering biofilm colonies, or smaller samples comprising only a few particles. The power and control unit 120 may also control the UV verification and sanitization abilities of device 100. Here, UV verification may refer to a different wavelength emitted by the nodes 101 to detect and biofilm and biofilm growth. The thickness of the biofilm may determine what wavelength is necessary for the sanitization application; this thickness may be inferred or calculated by the degree of luminescence originating from the biofilm. UV sanitization may be performed by emitting wavelengths that are predetermined by the power and control unit 120, or these wavelengths may be learned over time based on the device 101 readings and measurements. In one exemplary embodiment, a biofilm may grow due to a reduced flowrate through a holding tank. The location of the reduced flowrate and the biofilm may be determined by a lower level of luminescence. The luminescence may be transferred to the display screen 121 of the power and control unit 120 along with the node location and a reference map for the user.

FIG. 7 is a simple representation of one embodiment of the power and control unit 120. Additional embodiments may include different features as well as different feature layouts. In some embodiments, control of the device may be implemented in software in place of a physical controller. The power and control unit 120 may be equipped with controls for LED sources and narrow band pass filters for producing and capturing UV light with a desired wavelength or wavelengths. These controls (buttons 122-129) may enable UV and LED detection and sanitization of biofilm while the device is deployed. The power and control unit 120 may also include control of photomultiplier tubes for enhancing the emitted or captured signal etc., capable of changing the wavelength of the UV light produced necessary for killing biofilm and sanitizing surfaces. In one embodiment, the guide wires 116 may be controlled using a joystick 130 positioned on the power and control unit 120. In some embodiments, the power and control unit 120 may also be outfitted as a touchscreen, where some or all control features are available using touch screen functionality. An illumination control is provided for adjusting the strength of the UV lighting from all nodes 101 or a subset thereof. In addition, software and hardware control features are built into the power and control unit 120 to automatically reduce the overall output from the UV LED assemblies. This controls and mitigates image saturation during strong fluorescence. A scale bar displaying the strength of the fluorescence may also be displayed on the image display screen.

Data tracking using the power and control unit may comprise tracking an amount of biofilm. This amount may be used to output a model of the changing amount over time, as communicated via wireless or wired signal from the power and control unit to a display or processing unit of the user's choosing. Tracked data may include a temperature, composition, and/or turbidity value associated with the biofilm. It is contemplated that the output from the power and control device data tracking includes a mapping or quantified value of the biofilm detected by the device.

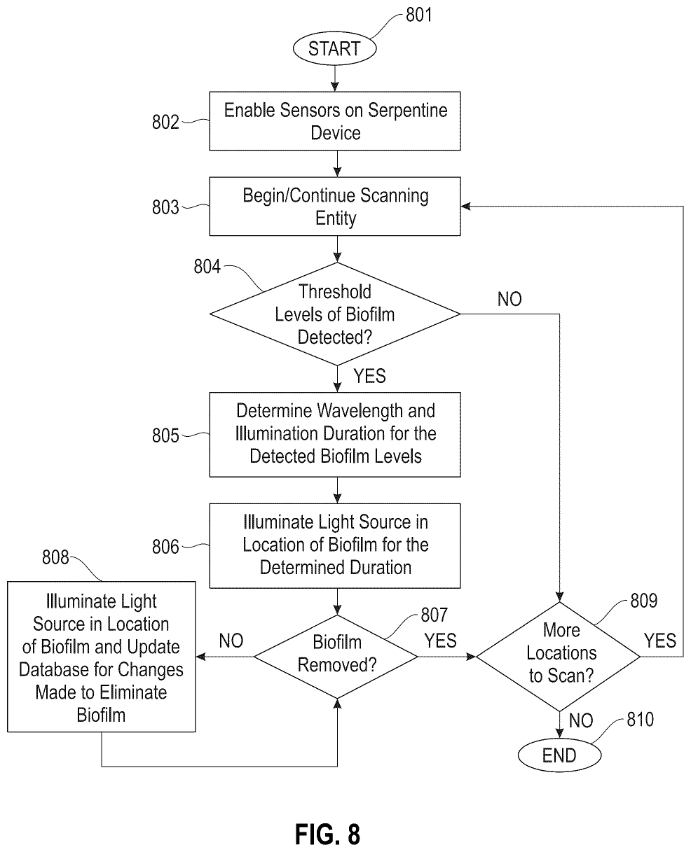

FIG. 8 is an operational flowchart illustrating a method 800 for the flexible serpentine device during deployment. In step 801, the device is powered on, in some embodiments by a power button on the power and control unit 120. The communication between the device and the power and control unit may be wirelessly communicated or communicated through a wired means. In step 802, the method may include enabling sensors on the serpentine device. This may comprise selecting one or more nodes as active, where active nodes are engaged for sensing biofilms and/or sanitizing biofilms. The one or more nodes may comprise each node present in the serpentine device, or a selected subsection of the nodes.

In step 803, the method may include beginning or continuing a scan of an entity. The entity may be an industrial material, a liquid vat used in processing, a healthcare-related instrument, or an HVAC system, for example. The scan may include moving the device along a predetermined path through the entity, or controlling the device as it is deployed throughout the entity. Control of the device may be achieved through using a joystick, or other suitable control of the guide wires controlling device movement. In some embodiments, the serpentine device moves via motor. In some embodiments, the serpentine device moves manually based on a user positioning. In some embodiments, the serpentine device may move iteratively through each activated node.

In step 804, the method may include determining a threshold level of biofilm detected. The threshold level may be predetermined and captured from a look-up table associated with the identified biofilm, where the control unit determines a category of biofilm based on one or more scan of the entity and uses that information to determine some characteristic of the biofilm that corresponds to the threshold. If the quantity or thickness of the detected biofilm is below the threshold, the control unit may send instructions to the device to move to another scanning location. If the quantity or thickness of the biofilm is above the threshold, the control unit may record a value associated with the quantity or thickness. In some embodiments, the threshold level may be determined by a preprogrammed library of reflective material index.

It is contemplated that the method 800 may include a noise elimination feature. This feature may be used in conjunction with the thresholding step 804. The threshold may be determined not only by the biofilm detected, but by the material scanned. For example,

In step 805, the method may include determining a wavelength and illumination duration for the detected biofilm level. If the detected biofilm level is above the predetermined threshold, the wavelength and illumination may be determined from checking an associated database, and/or by calculating the necessary wavelength and illumination based on the threshold value.

In step 806, the method may include illuminating a light source in the location of the biofilm for a determined illumination duration.

In step 807, the method may include determining whether the biofilm has been removed. If the device and control unit determines that the biofilm is adequately removed and no longer necessitates the application of the wavelength and illumination from the device, the device and control unit may then review other locations within the entity to determine whether there are more locations to scan (step 809). If the device and control unit determine there are additional locations to scan in step 809, the method may include returning to step 803 to begin another scan or continue scanning the entity.

If, however, the biofilm is determined to have not been adequately removed from the entity in step 807, the method may progress to step 808. In step 808, the method may include illuminating the light source in the location of the biofilm and updating a related database for changes made by the device to eliminate the biofilm. Changes may be made to the illumination duration and/or wavelength, depending on whether some measurement of the biofilm has been removed. A notification to the user may be generated if multiple attempts to remove the biofilm have been made and the biofilm is not removed.

Once step 808 is completed, the method may return to step 807 as part of a feedback loop to determine that the biofilm is adequately removed from the entity. After determining that the biofilm is adequately removed, the method will end at step 810. The output of the method may include a visual representation of biofilm over exposure time to the UV LED source, luminescence after time, wavelength used compared to number of reflective particles, or other suitable analysis.

### Use Case 1: In Situ Inspection of Liquid Storage Tanks

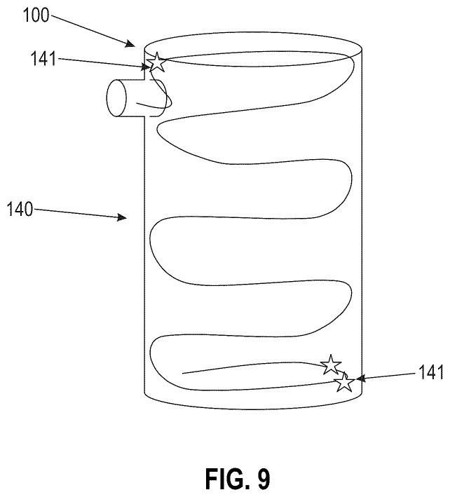

FIG. 9 illustrates an example of the device 100 used in in situ inspection of a liquid storage tank 140. In this exemplary case, the device 100 may be deployed as a long-term monitoring system for biofilm formation within a liquid storage tank 140. A flexible strand of detector nodes, such as nodes 101, may be installed around the outside of tank 140. A contamination location marker 141 may be indicated at a starting point of the device 100, corresponding to an initial scan or start point of the device.

The device 100 may be of any length, so long as there is no degradation of signal between the device 100 and the power and control unit. The device length may correspond to the size of holding tank 140, or be longer or shorter as desired. As shown in FIG. 9, the device may cover the entirety of the tank 140 through its serpentine looping, such that each possible location for the formation of biofilm is readable by the device 100. The device 100 should be installed such that individual nodes make contact with the corners and sidewalls of the holding tank 140, as these areas are particularly susceptible to the formation of biofilm.

If biofilm is detected by the device 100, an alert may sound along with a display and/or readout of the node that detected the biofilm and its location in situ. If biofilm is concurrently detected at multiple nodes, the relative location of the biofilm may be displayed to the user on a display screen. It is contemplated that a map may also be output to a display screen, where levels of biofilm formation are likewise shown in the context of the holding tank 140. A contamination location marker 141 may be used to indicate areas of particular interest, and to ensure that each area is highlighted by a related node.

In this exemplary application, a port with waterproof seals may be required to enable transmission of power and signal to and from the device. Air-and water-tight seals may be used to protect cabling. It is contemplated that wireless communication between the device and a corresponding power and control unit would be of particular usefulness, such as communication via Bluetooth, WiFi, or short-wave radio frequencies.

Applications of the device within holding containers can include general cases of inspection and sanitization. This application may include stored components such as hoses, and other components that cannot be sanitized using liquids. Application in the industrial setting of water contamination, cooling components, air conditioning, and ventilation for residential and industrial complexes may be useful as well, as these components are often difficult to access and sanitize using alternative methods. Further, it is contemplated that the biofilm detection and sanitization method may be used in the sanitization and monitoring of food service industry spaces including refrigeration, food storage, and preparation spaces. Additional applications may include hospital, factory, and food manufacturing industry use.

### Use Case 2: Bronchoscopy

It is contemplated that the flexible serpentine device may be used to enrich surgical knowledge within an artificial intelligence corpus. Use of the device may help surgeons and associated caregivers to better understand and visualize the effect of biofilm within a surgical context.

Bronchoscopy is a procedure that lets doctors look at your lungs and air passages. It's usually performed by a doctor who specializes in lung disorders (a pulmonologist). During bronchoscopy, a thin tube (bronchoscope) is passed through your nose or mouth, down your throat and into your lungs. Bronchoscopy is most commonly performed using a flexible bronchoscope. However, in certain situations, such as if there's a lot of bleeding in your lungs or a large object is stuck in your airway, a rigid bronchoscope may be needed. Common reasons for needing bronchoscopy are a persistent cough, infection or something unusual seen on a chest X-ray or other test. Bronchoscopy can also be used to obtain samples of mucus or tissue, to remove foreign bodies or other blockages from the airways or lungs, or to provide treatment for lung problems.

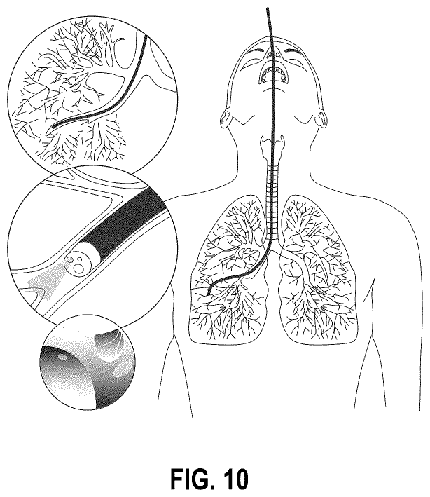

As shown in FIG. 10, it is contemplated that the flexible serpentine device can be used within medical applications such as bronchoscopy. As embodiments of the present disclosure include the ability to illuminate biofilm with a unique method of dynamic fluorescent light exposure, this ability may be capitalized on through use of the device within the body to better visualize lung and air passages. Further, the iterative management of categorical reference to different types of biofilms may allow pulmonologists and other healthcare providers wider access to encyclopedic knowledge otherwise unavailable.

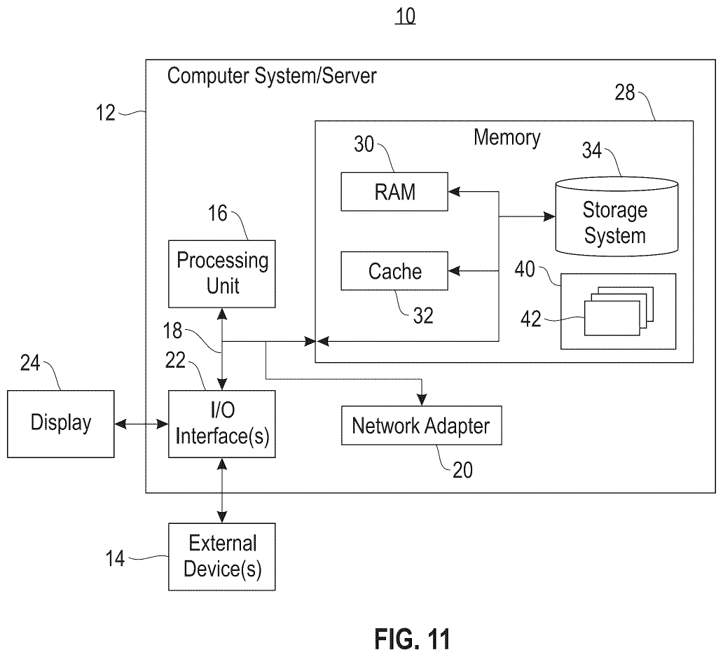

Referring now to FIG. 11, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 11, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A biofilm detection device comprising:
a plurality of sensing nodes, each of the plurality of sensing nodes comprising:
one or more probes spaced around and defining a center lumen of the sensing node and having a surface directed outwardly from the center lumen, each of the one or more probes comprising:
a quartz window disposed in the surface; and
a fiber optic element disposed in optical communication with the quartz window;
one or more guide wire ports running parallel to the center lumen;
a plurality of swivel joints, each of the plurality of swivel joints interconnecting adjacent pairs of the plurality of sensing nodes; and
one or more guide wires extending through the guide wire ports.

2. The device of claim 1, further comprising a hollow backbone carrying fiber optic cabling from the sensing nodes in communication with a control unit.

3. The device of claim 1, wherein the probes are air- or water-tight.

4. The device of claim 1, wherein the quartz window comprises a UV grade quartz, and transmits UV light ranging from 180 nm to 400 nm.

5. The device of claim 1, wherein the one or more probes further comprises one or more light emitting diodes (LED).

6. The device of claim 1, wherein each sensing node of the plurality of sensing nodes comprises a plurality of optically transparent windows configured to provide 360 degree light emission and sensing.

7. The device of claim 1, wherein each sensing node of the plurality of sensing nodes is independently operative.

8. The device of claim 1, wherein each sensing node of the plurality of sensing nodes further comprises a series of open ports positioned symmetrically about the center lumen.

9. The device of claim 8, wherein each quartz window is detachably attached to one or more of the open ports by a latch.

10. The device of claim 1, wherein the fiber optic element is configured to receive emitted fluorescence from biofilm.

11. The device of claim 1, wherein the plurality of sensing nodes is configured to emit fluorescence to thereby sanitize biofilm.

12. The device of claim 1, wherein the plurality of sensing nodes is operatively connected to a control unit via one or more wires extending through the center lumen.

13. The device of claim 1, wherein the swivel joints comprise hollow ball joints.

14. The device of claim 1, wherein the plurality of sensing nodes is operatively connected to a control unit via a wireless network.

15. A method of detecting biofilm, the method comprising:
activating one or more sensing nodes on a biofilm detection device, the biofilm detection device comprising at least one sensing node with an illumination source;
scanning an entity using the biofilm detection device;
detecting a threshold level of a biofilm within the entity;
determining a wavelength and an illumination duration for the threshold level of the biofilm;
illuminating, by the illumination source, a location of biofilm within the entity for a determined illumination duration;
determining the biofilm is removed from the location; and
outputting a report of an end level of biofilm within the entity.

16. The method of claim 15, wherein the biofilm detection device comprises:
a plurality of sensing nodes, each of the plurality of sensing nodes comprising:
one or more probes spaced around and defining a center lumen of the sensing node and having a surface directed outwardly from the center lumen, each of the one or more probes comprising:
a quartz window disposed in the surface; and
a fiber optic element disposed in optical communication with the quartz window;
one or more guide wire ports running parallel to the center lumen;
a plurality of swivel joints, each of the plurality of swivel joints interconnecting adjacent pairs of the plurality of sensing nodes; and
one or more guide wires extending through the guide wire ports.

17. The method of claim 15, further comprising:
determining a biofilm remains in the location;
illuminating, by the illumination source, a location of biofilm for an additional illumination duration;
determining the biofilm is removed from the location; and
updating the report.

18. The method of claim 15, further comprising updating a database associated with the entity, wherein the database includes changes made to eliminate biofilm within the entity.

19. The method of claim 15, further comprising communicating the report to a display.

20. The method of claim 15, wherein scanning the entity comprises a lateral, 360-degree biofilm sensing and sanitizing by the one or more nodes.

21. The method of claim 20, further comprising inducing a fluorescent emission from the biofilm by the one or more nodes.

22. The method of claim 21, further comprising storing and recording the fluorescent emission as the entire entity is scanned.
